(19) United States
(12) Patent Application Publication
(10) Pub. No.: US 2003/0068790 A1
(43) Pub. Date: Apr. 10, 2003

(54) Enzymatically catalysed process

(76) Inventors: Robert Wilhelm Van Gemert, S N Arnhem (NL); Folkert Petrus Cuperus, MC Staphorst (NL)

Correspondence Address: Intellectual Property Group, Pillsbury Winthrop LLP, 1600 Tysons Boulevard, McLean, VA, 22102, US

(21) Appl. No.: 10/138,778

(22) Filed: May 6, 2002

Related U.S. Application Data
(63) Continuation of application No. 09/367,384, filed on Aug. 13, 1999.
Application No. 09/367,384 is a 371 of international application No. PCT/EP98/00726, filed on Feb. 10, 1998.

(30) Foreign Application Priority Data
Feb. 14, 1997 (EP) 97200424.6

(51) Int. Cl. C12P 7/64

(52) U.S. Cl. 435/134

(57) ABSTRACT

Enzymatically catalysed process, in which reaction water formed is continuously removed through a non-porous, water-permeable membrane. The enzyme is immobilised on the membrane. The invention further relates to a non-porous, water permeable membrane, with immobilised thereon an enzyme, as well as to a reactor in which such a membrane is used. Lipases are preferred enzymes in the above.

The present invention relates to an enzymatically catalysed process, in which water is liberated as the reaction proceeds. More in particular, this invention relates to such a process in which the water formed is continuously removed. The invention further relates to a membrane, with immobilised thereon an enzyme, as well as to a reactor in which such a membrane is used.

There are various reactions known which are catalysed by enzymes and in which reactions water is produced as one of the reaction products. Examples of such reactions are esterifications of alcohols and acids using lipases, etherifications of mono-saccharides to polysaccharides using carbo-hydrases, formation of amides from amino acids using proteases, etcetera. In many cases, the enzyme catalyst employed is immobilized on a carrier, which is often a porous particulate material. The enzyme is then immobilized on the surface of the carrier material (mainly inside the pores). For continuous processing, said particulate porous carrier is often contained in a packed bed. An example of a frequently used carrier material is Accurel (ex Akzo Nobel).

When the reaction proceeds in a system wherein the enzyme is immobilized according to the above, a common encountered problem is accumulation of one or more of the reaction products inside the pores of the carrier material, thus blocking the pores and preventing the reactants from entering the pores, leading to a steady decrease of the activity and/or productivity. This accumulation frequently occurs with water, in reaction systems in which water is liberated, such as described above. This poses a problem in designing the reactor lay-out.

Also, many of the reactions as set out above are equilibrium reactions, and as a consequence, only proceed to completion upon removal of one or more of the reaction products.

Many investigations have been undertaken to design a continuous, enzymatically catalysed process in which water is removed as the reaction proceeds, so as to prevent reduction in activity/productivity, and/or to achieve shifting of the reaction equilibrium to the more desired side, but none of these processes have proven to be entirely satisfactory.

A solution which has been proposed by Van der Padt et al (Journal of Membrane Science, 80 199-208 (1993)) for a lipase-catalysed esterification involves continuous processing in a reactor, in which water is removed through a porous membrane, on which membrane the enzyme is immobilized. The water is removed by passing air or an organic medium over the outer layer of the membrane, having a lower water activity than at the inner side (is reactor side) of the membrane. Although this may partly work, it suffers from the disadvantage that the driving force applied cannot be very large. Applying a reduced (absolute) pressure on the outer side of the membrane increases water flux over the porous membrane, but besides water, part of the reaction medium, involving organic compounds (e.g. fatty acids, alcohols) are also leaking through the pores of the membrane. Such a system is of limited use in practice.

Hence, there was a need for an improved enzymatically catalysed process, in which water is liberated as the reaction proceeds, and in which water is removed in an effective manner as the reaction proceeds. Preferably, such a process should be suitable to be operated in a (semi-) continuous manner.

It has now been found that the above objective(s) can be met by an enzymatically catalysed process in which process water is liberated, wherein at least part of the water is removed through means, the means comprising a layer of immobilized enzyme and a layer of a non-porous, water-permeable membrane.

In other words, the invention relates to a process in which means are installed, which means serve both as support for the enzymes and for separating water from the reactor through a non-porous water-permeable membrane.

The “non-porous, water permeable membrane” above (hereafter referred to as the membrane), is herein to be understood to be a sheet-like material through which water can pass, but which does not contain pores extending through the membrane. This is an important feature, since if pores are present, one cannot use a too large pressure difference as the driving force for removing water, as this increases the risk of other components besides water to be transported through the membrane. In general, the pores in porous membranes are that large that besides water molecules small alcohols and fatty acids may pass as well, as long as the pressure difference is large enough. The passage of water through non-porous, water-permeable membranes by applying a driving force is known in the art, and can be explained by the dissolution-diffusion model.

The water can be removed from the reactor through the membrane in any conventional way, a long as a driving force is established. This can suitable be achieved by applying a difference in the absolute pressures at the inner- and the outer side of the membrane. Preferably, this is achieved by reducing the pressure on the outer side of the membrane to sub-atmospheric, since the pressure at the inner side of the membrane (i.e. in the reactor) is generally atmospheric. In an alternative arrangement, the pressure on the inside may be increased. Preferably, for efficient water removal by applying a pressure difference, the pressures should be chosen such that the (absolute) pressure at the outer side of the means is less than the partial water pressure at the inner side of the means. More preferably, the (absolute) pressure at the outer side of the means is 60% or less of the partial water pressure at the inner side of the means. Most preferably, the above percentage is 30%.

Additional benefits of using the process according to the invention may include an increased enzyme activity (i.e. yield per gramme of enzyme), when compared with conventional immobilisation techniques. An increase of 10%, or even 20% may be possible. Furthermore, separation of water may be more effective than when using a process in which the enzyme is immobilised in e.g. a packed bed, instead of in or on the membrane. This is expressed as a smaller membrane surface that may be needed (e.g. 20% to 40% smaller).

Preferably, the reaction components and products besides water, as well as the enzyme, should be retained in the system, and hence, it is preferred that the non-porous, water-permeable membrane is impermeable for these substances, in particular for organic substances. Such membranes are known in the art of chemical processing and separation. In order that the membrane lasts long enough, the membrane employed must be reasonably stable towards the reactants and the reaction products (e.g. alcohols, organic acids, esters, water, solutions of sugars, amino acids and proteins, etcetera).

An advantage of the process according to the invention is that the process can be operated at water-saturated conditions, without the productivity being affected.

It is advantageous from a processing point of view if all or the major part of the enzyme is immobilized on the membrane, although there may be some free enzyme floating around in the reaction medium. This is not detrimental to the process. It is generally also advantageous if the complete membrane surface area is utilized for immobilizing the enzyme.

The enzyme may be immobilized on the membrane in various ways. In general, the non-porous, water-permeable membranes comprise a porous support layer with attached thereto a toplayer which is non-porous and water-permeable, to form together a non-porous, water permeable membrane (see FIGS. 1A and 1C: the composite membrane).

In the most simple arrangement the enzyme may be directly immobilized on the top layer of the non-porous, water-permeable membrane, as is exemplified in FIG. 1A (enzyme given in solid black). The reaction medium is on the enzyme side, the water is removed through the toplayer, through the porous support. (In FIG. 1A the water flow is from top to bottom.)

In an alternative arrangement the enzyme may be immobilized on the reversed side of the membrane (i.e. the porous support), whilst the non-porous top layer is facing outwards and the porous support facing the reaction medium (FIG. 1B).

In yet a further alternative, the enzyme may be immobilized on a separate porous support membrane (e.g. a glassfibre membrane, a polypropylene membrane, etcetera), which is subsequently attached to a non-porous, water-permeable membrane (see FIG. 1C).

In order to immobilize the enzymes to the membrane various techniques as known in the art may be employed.

The arrangements as set out above may comprise more layers, e.g. a supportive membrane or a protective layer. Generally, such extra layers or support membranes are permeable not only for water, but also for other liquid components. In that case, the non-porous, water-permeable membrane ensures water can be removed from the reaction medium, whilst retaining substantially all of the other reaction components.

In some applications (depending e.g. on the type of membrane used and the type of enzyme employed) it may be desired to subject the membrane to a pre-treatment first, before adhering the enzymes, so as to ensure an improved adherence. Examples of such pre-treatment are silylization of the membrane, or the application of a silica top layer or pre-immobilization of a protein.

The amount of enzyme which is to be immobilized on the membrane carrier (i.e. the enzyme loading) depends very much upon the type of enzyme used, purity, the activity, the reaction conditions, etcetera, and can be determined by the person skilled in the art using a reasonable amount of experimentation. The layer of immobilized enzyme on the membrane may have a thickness of between 2-100 nm, preferably 5-20 nm, more preferably between 10-15 nm (all average layer thicknesses). It may be clear that besides the immobilized enzyme, there may be some free enzyme floating around in the reaction medium. This is not detrimental to the process.

When operating the process according to the invention, the amount of water present in the reaction system should be carefully controlled: too little water may cause damage to the enzyme (e.g. because of denaturation). On the other hand (but this is less critical) too much water may be disadvantageous when high conversions are desired. Therefore, it is preferred that the total amount of water present in the reaction system should be such that the thermodynamic water activity is at least 0.01, preferably 0.10, but more preferably 0.25. Although this system may work when saturated with water (i.e. water activity of 1.0), an upper limit of 0.7, preferably 0.5 is preferred. The rate of water removal can be controlled in a way as is known for the membranes as such (i.e. without the immobilized enzyme).

The process according to the invention is especially suitable for application in continuous or semi-continuous processes.

The process according to the invention can be employed well for the lipase catalysed esterification of alcohols with carbon acids. Regarding the latter, fatty acid esterification is preferred. Preferably, such a fatty acid has between 6 and 24 carbon atoms. The fatty acids can be either straight or branched chain, saturated or unsaturated fatty acids. Lauric acid, oleic acid, stearic acid and iso-stearic acid are preferred. Regarding the alcohol part, the process is especially useful for the esterification of fatty alcohols with acids. Such alcohols may have chain lengths of between 6 and 24 carbon atoms. Preferably, such an alcohol is 2-ethyl hexanol.

In between two process cycles, the membrane may be cleaned, whereafter a fresh enzyme layer may be immobilized. Cleaning of the membrane can be effected by e.g. using an alkaline solution.

For carrying out the process according to the invention, (bio)reactors are employed for performing enzymatically catalysed reactions, wherein the at least part of the enzyme is immobilized on a non-porous, water-permeable membrane. Preferably, water is removed through said membrane during the process. Lipases are especially suitable for application on the membrane in the reactor as set out above.

The invention also comprises a non-porous, water-permeable membrane, characterized in that en enzyme is immobilized on the membrane. Preferably, the enzyme comprises a lipase.

The invention is further illustrated by the following examples, which are not to be understood as limiting the invention thereto.

### EXAMPLE 1

Materials

As a model was chosen the enzymatic esterification of oleic acid with 2-ethyl-1-hexanol to iso-octyloleate. The reaction was catalysed using two different lipases: Lipomax (a lipase ex Pseudomonas alcaligenes, as can be obtained from Genencor, Delft, the Netherlands) and Lipolase (a lipase ex Humicola, as can be obtained from NOVO, Denmark).

The membranes used to immobilize the enzymes are coded GFT-2002, (as can be obtained from GFT Deutsche Carbone AG, Germany) and a ceramic membrane, (as can be obtained form F. M. Velterop BV, Enschede, the Netherlands). The GFT-2002 membrane used in combination with Lipomax had a surface area of 2.92×10⁻³³ m², the GFT-2002 membrane used in combination with the Lipolase enzyme had a surface area of 12.3×10⁻³ m₂, and the ceramic membrane had a surface area of 1.26×10⁻³ m². In one instance, the GFT-2002 membrane was used in a reverse position, which made a higher enzyme loading possible. For the other examples, the membranes were used in the direction as suggested by the manufacturers.

The oleic acid (Priolene 6905, ex Unichema International, Gouda, the Netherlands). The 2-ethyl-1-hexanol used was of high purity.

Immobilization

The enzymes were immobilized on the membranes using the following procedure. For all immobilization procedures, a “tris”-buffer (tris(hydroxymethyl)-aminomethane, C₄H₁₁NO₃) buffer solution was prepared by dissolving 0.05 mol “tris” in 400 ml water (6 g/l), which resulted in a solution having pH 10. The pH was decreased to 8 by further adding a concentrated solution of HCl (2M). About 5.8 ml was added.

Prior to immobilisation, each fresh membrane was treated with a saturated 2-ethyl-1-hexanol solution, in order to avoid too rapid swelling of the membrane.

Immobilization of Lipomax (ex Pseudomonas alcaligenes) on GFT-2002 (both normal and reversed): approximately 0.145 gram of the enzyme as obtained from the manufacturer (i.e. dry form) was dissolved in 1000 ml of said tris-buffer solution, and 0.1 gram of Triton X100 surfactant was added. 750 ml of this solution was flushed for 18 hours over the GFT-2002 membrane used to immobilize the enzyme. The flushing operation was carried out in the same manner as is described below under the header “reaction system”. The immobilization efficiency was tested with a tributyrin test of the remaining enzyme solution, from which the enzyme loading was calculated.

Immobilization of Lipolase (ex Humicola) on ceramic membrane: 10.64 gram of the commercial enzyme solution (3% by weight of enzyme) was added to 400 ml of said tris-buffer. This solution was flushed for 11 hours over a ceramic membrane as mentioned above, having an area of 1.26×10⁻³ m². The immobilization efficiency was tested with a tributyrin test of the remaining enzyme solution, from which the enzyme loading was calculated.

Immobilization of Lipolase on GFT-2002 membrane: 0.227 g of commercial enzyme solution (3% by weight of enzyme) was added to 400 ml of said tris-buffer. This solution was flushed for 18 hours over a GFT-2002 membrane as mentioned above, having an area of 12.3×10⁻³ m². The immobilization efficiency was tested as above.

Reaction system

The membranes so prepared with lipases immobilized thereon were tested in a reaction system for the preparation of oleic acid with 2-ethyl-1-hexanol to iso-octyloleate. The membranes with immobilized enzymes were used as flat modules. Prior to the reaction, the membranes were flushed 5 times with pure 2-ethyl-1-hexanol, 3 times at room temperature, once at 50° C., and once at 60° C. Directly thereafter, the reaction mixture was circulated through the membrane. The reactor arrangement used was a semi-batch operated set-up.

From a storage flask, the reaction mixture is pumped at a flow rate of about 50 ml/min through the flat membrane module, and recycled through the 250 ml flask. The storage flask was continuously stirred and temperature controlled at a set temperature of 60° C.

The reaction components 2-ethyl-1-hexanol and oleic acid were used in a molar ration of 1:1. In experiment 1 (see table 1) the amount of oleic acid was 0.4 mol, in experiments 2, 3 and 4 this was 0.13, 0.5 and 0.5 mol, respectively.

Reactions were carried out until a conversion of 99% was achieved. At regular intervals, samples were taken, from which the data in table 1 have been calculated.

The sampled product was analysed after methylation of the free fatty acids using diazomethane. After dilution with hexane, the samples were analyzed using GLC (CP-Sil column).

Results

The results from the experiments can be seen in table 1. From the concentrations measured, the activities and productivities have been calculated.

### EXAMPLE 2

In this example, the enzyme was immobilised on a membrane having large pores (Accurel sheet, ex Akzo, pores of about 0.2 micrometers).

In order to achieve this, 3.702 g of Lipomax was dissolved in 1000 ml Tris buffer solution of pH 8.0 (see example 1 for details). Using a Millipore MF-filter having pores of 0.45 micron, 0.739 Undissolved enzyme was filtered off. The resulting solution (2.963 g of enzyme in 1000 ml Tris buffer) was immobilized on a membrane as set out above having a surface area of 2.922×10⁻³ m². This was done by flushing 450 ml of the above mentioned enzyme solution for 24 hours over the membrane (pressure 1.5 bar). Said membrane was treated first with ethanol to fill all the pores. Testing with tributyrin showed that the membrane set so prepared had an enzyme loading of 325 g/rm² (0.952 g of enzyme on 2.922×10⁻³ m²).

After immobilisation of the enzyme on the support membrane, it was covered with a water permeable membrane (GFT 2002, as above, surface area 2.922×10⁻³ m², in such a way that the enzyme is enclosed by two membranes, one with large pores, and one with small pores. Thereafter, said arrangement was tested along the lines as described in example 1.

The activity measured at 50% conversion was 4.5 mmol fatty acid/g.h, and the productivity was 1.47 mol ester/h.m².

TABLE 1
activities and productivities for the reaction as specified,
for various combinations of lipase and supporting membrane

| | | | Enzyme | Activity (50% | Productivity | |
|---|---|---|---|---|---|---|
| | | buffer | loading | conv.) mmol | mol ester/H.m² | |
| Support | Enzyme | pH | g/m² | FA/g.enz./h | 50% conv. | 95% conv. |
| GFT-2002 | Pseudomonas alcaligenes | 8.2 | 5.37 | 106 | 0.56 | 0.33 |
| | lipase (Lipomax) | | | | | |
| GFT-2002 | Pseudomonas alcaligenes | 8.5 | 84.9 | 19.6 | 1.66 | 1.03 |
| (reversed) | lipase (Lipomax) | | | | | |
| GFT-2002 | Humicola | 8 | 0.3 | 207 | 0.09 | n.d. |
| | lipase (Lipolase) | | | | | |
| Ceramic | Humicola | 8 | 49.44 | 20.7 | 1.02 | n.d. |
| | lipase (Lipolase) | | | | | |

1. Enzymatically catalysed process in which process water is liberated, wherein at least part of the water is removed through means, the means comprising a layer of immobilized enzyme and a non-porous, water-permeable membrane.

2. Process according to claim 1, characterized in that the membrane is impermeable for organic substances.

3. Process according to claim 1 or 2, characterized in that the water is removed through the means by applying a driving force in the form of a pressure difference.

4. Process according to claim 3, characterized in that the pressure at the outer side of the means is lower than atmospheric pressure.

5. Process according to claim 4, characterized in that the absolute pressure at the outer side of the means is less than the partial pressure of the water at the inner side of the means.

6. Process according to any of claims 1-5, characterized in that the process is carried out in a (semi-) continuous manner.

7. Process according to any of claims 1-6, characterized in that the enzymatic catalyst comprises a lipase.

8. Process according to any of claims 1-7, characterized in that the reactant mixture comprises an alcohol and a fatty acid.

9. Process according to claim 8, characterized in that the fatty acid has between 6 and 24 carbon atoms.

10. Process according to claim 8 or 9, characterized in that the alcohol comprises a fatty alcohol having between 6 and 24 carbon atoms.

11. Process according to claim 10, characterized in that the alcohol comprises 2-ethyl-1-hexanol.

12. Process according to any of claims 1-11, characterized in that the water activity in the reactor is higher than 0.01.

13. Process according to any of claims 1-12, characterized in that the layer of immobilized enzyme on the membrane in a layer thickness of between 2-1000 nm, preferably 5-20 nm.

14. Reactor for performing reactions catalysed by an enzyme, characterized in that at least part of the enzyme is immobilized on a non-porous, water-permeable membrane.

15. Reactor according to claim 14, characterized in that water is removed through the membrane during the process which is performed in the reactor.

16. Reactor according to claim 14 or 15, characterized in that the enzyme comprises a lipase.

17. Non-porous, water-permeable membrane, characterized in that an enzyme is immobilized on the membrane.

18. Membrane according to claim 17, characterized in that the enzyme comprises a lipase.
